(19) United States
(12) Patent Application Publication
(10) Pub. No.: US 2012/0108678 A1
(43) Pub. Date: May 3, 2012

(54) BRANCHED COPOLYMERS, COMPOSITIONS AND USES

(75) Inventors: Paul Hugh Findlay, Liverpool (GB); Brodyck James Lachlan Royles, Liverpool (GB); Neil John Simpson, Liverpool (GB); Sharon Todd, Liverpool (GB); Steven Paul Rannard, Liverpool (GB); Jonathan Victor Mark Weaver, Liverpool (GB); Roselyne Marie Andree Baudry, Liverpool (GB)

(73) Assignee: UNILEVER PLC, London (GB)

(21) Appl. No.: 13/148,471

(22) PCT Filed: Feb. 9, 2010
(86) PCT No.: PCT/GB2010/000227

§ 371 Date: Aug. 8, 2011

(30) Foreign Application Priority Data
Feb. 9, 2009 (GB) 0902052.0

(51) Int. Cl. A61K 47/32; C08L 47/00; C09D 147/00; C09J 147/00; C09K 15/12; C02F 5/00; C10M 107/42; C10M 107/28; C09K 5/00; C09K 15/28; C08F 236/20; C09D 11/00

(52) U.S. Cl. 514/772.5; 526/320; 526/263; 526/312; 524/548; 524/555; 524/558; 514/772.6; 252/180; 508/264; 508/472; 508/470; 252/78.1; 252/402; 252/406

(57) ABSTRACT

The present invention relates to a branched copolymer obtainable by an addition polymerisation process and uses and compositions thereof comprising:
i) a residue of at least one monofunctional monomer comprising one polymerisable double bond per molecule and a molecular weight of less than 1000 Daltons;
ii) a residue of at least one multifunctional monomer comprising at least two polymerisable double bonds per molecule and a molecular weight of less than 1000 Daltons; and wherein the end termini of the copolymer chains comprise one or more of a residue of a chain transfer agent; an initiator or a terminal group derived from a termination reaction;
wherein;
   the molar ratio of the monofunctional monomer to multifunctional monomer is between 50:1 to 2.5:1 respectively; and
   wherein the hydrophilic component is comprised of at least 1 mole % of a combination of a monofunctional monomer, and a multifunctional monomer and/or a chain transfer agent when compared to the total content of monofunctional monomer which is/are comprised of hydrophilic component each with a solubility of 0.18 w/w % in water at 20° C.; and
   wherein the residue of the at least one monofunctional monomer with a molecular weight of less than 1000 daltons is selected from the group comprising:
   vinyl acids, vinyl acid ester, vinyl aryl compounds, vinyl acid anhydrides, vinyl amides, vinyl ethers, vinyl amines, vinyl aryl amines, vinyl nitriles, vinyl ketones, and derivatives thereof;
   hydroxyl-containing monomers and monomers which can be post-reacted to form hydroxyl groups;
   acid-containing or acid functional monomers;
   zwitterionic monomers;
   quaternised amino monomers, oligomeric monomers; and corresponding allyl monomers of the aforesaid vinyl monomers.

### TECHNICAL FIELD

The present invention relates to certain branched addition copolymers which may be water-soluble or water dispersible, a method for their preparation, compositions containing such copolymers and their use in for example aqueous media or non-aqueous media.

More specifically, the present invention relates to certain branched addition copolymers wherein the copolymer comprises a hydrophilic component. Even more specifically, the present invention relates to certain branched addition copolymers wherein the copolymer comprises a hydrophilic component derived from a combination of at least 1 mole % hydrophilic monofunctional monomer and hydrophilic multifunctional monomer and/or hydrophilic chain transfer agent based on the total monofunctional monomer content.

The copolymers of the present invention find particular application where copolymers with a hydrophilic residue are required.

### BACKGROUND OF THE INVENTION

Branched polymers are polymer molecules of a finite size which are branched. Branched polymers differ from crosslinked polymer networks which tend towards an infinite size having interconnected molecules and which are generally not soluble in a solvent. In some instances, branched polymers have advantageous properties when compared to analogous linear polymers. For instance, solutions of branched polymers are normally less viscous than solutions of analogous linear polymers. Moreover, higher molecular weights of branched copolymers can be solubilised more easily than those of corresponding linear polymers of a comparable molecular weight. In addition, branched polymers tend to have more end groups than a linear polymer and therefore generally exhibit strong surface-modification properties. Thus, branched polymers are useful components of many compositions utilised in a variety of fields but are often difficult to manufacture in sufficient quantities to be commercial useful.

Branched polymers are usually prepared by means of a step-growth mechanism via the polycondensation of suitable monomers. However, the choice of monomers to be utilised is usually limited by the required chemical functionality of the resulting polymer and the molecular weight. In addition polymerisation, a one-step process can be employed in which a polyfunctional monomer is used to provide functionality in the polymer chain from which polymer branches may grow. However, a limitation on the use of conventional one-step processes is that the amount of polyfunctional monomer must be carefully controlled, usually to substantially less than 0.5% w/w in order to avoid extensive cross-linking of the polymer and the formation of insoluble gels. It is also often difficult to avoid crosslinking using this method, especially in the absence of a solvent as diluent and/or at high conversion of monomer to polymer.

WO 99/46301 (granted as EP1062248) discloses a method of preparing a branched polymer comprising the steps of mixing together a monofunctional vinylic monomer with from 0.3 to 100% w/w (of the weight of the monofunctional monomer) of a multifunctional vinylic monomer and from 0.0001 to 50% w/w (of the weight of the monofunctional monomer) of a chain transfer agent and optionally a free-radical polymerisation initiator and thereafter reacting said mixture to form a copolymer wherein the molecular weight of the polymer is in the range 2 to 200 kDa. The examples in WO 99/46301 describe the preparation of primarily hydrophobic polymers and, in particular, polymers wherein methyl methacrylate constitutes the monofunctional monomer. These polymers are useful as components of surface coatings and inks or as moulding resins.

WO 99/46310 (granted as EP1062258) describes a method of preparing a branched polymer which includes at least one polymerisable double bond comprising the steps of mixing together at least one monofunctional monomer having one polymerisable double bond per molecule with from 0.3 to 100% w/w (of weight of the monofunctional monomer) of a polyfunctional monomer having at least two polymerisable double bonds per molecule and from 0.0001 to 50% w/w (of the weight of a monofunctional monomer) of a chain transfer agent and optionally a free-radical polymerisation initiator. A key feature of WO 99/46310 is the termination of the polymerisation when less than 99% of the polymerisable double bonds arising from the monofunctional monomer have been reacted.

WO 02/34793 discloses a copolymer composition comprising a copolymer derived from at least one unsaturated carboxylic acid monomer, at least one hydrophobic monomer, a hydrophobic chain transfer agent, a crosslinking agent, and, optionally, a steric stabiliser. The copolymer composition acts as a rheology modifier in that it provides increased viscosity in aqueous electrolyte-containing environments.

U.S. Pat. No. 5,767,211 describes the synthesis of multi-functional hyperbranched polymers by free radical polymerization of di- or tri-vinyl monomers in the presence of a chain transfer catalyst and a non-peroxide free radical initiator. The polymers are useful for automotive coatings and for photopolymerization applications.

US 2004/063880 discloses branched polymers prepared by mixing together monofunctional vinylic monomers with from 0.3 to 100% w/w of polyfunctional vinylic monomer and from 0.0001 to 50% w/w of chain transfer agent and thereafter reacting the mixture to form a polymer. The resulting branched polymers find application as components of surface coatings and inks as well as molding resins.

U.S. Pat. No. 5,496,896 relates to a curable composition containing as component A) compounds with at least two activated double bonds (I), these being α,β-unsaturated carbonyl compounds, α,β-unsaturated carboxylic acid esters or α,β-unsaturated nitriles, and compounds B) which contain at least two active hydrogen atoms or at least one active hydrogen atom and at least one group with an active hydrogen atom, and customery additives, catalysts, pigments if appropriate and an organic solvent.

U.S. Pat. No. 5,962,613 details the synthesis of water-soluble copolymers which are obtainable by the free-radical polymerisation of from 10 to 99.5% by weight of at least one vinylimidazole, 0 to 89.5% by weight of other copolymerisable monoethylenically unsaturated monomers and, between 0.5 and 30% by weight of at least one monomer which acts as a cross-linker and has at least two non-conjugated ethylenic double bonds in water and/or polar organic solvents in the presence of polymerisation regulators, using from 0.1 to 5 parts by weight of polymerisation regulator per 1 part by weight of crosslinker and their use as additives for detergents.

US 2003/187166 relates to partially branched polymers having a number-average molecular weight Mn in the range of from 500 to 20,000 Daltons and synthesized from ethylenically unsaturated monomers including from 80 to 99.9% by weight of monoethylenically unsaturated monomers A and from 0.1 to 20% by weight of monomers B containing at least two non-conjugated ethylenically unsaturated double bonds, wherein the weight fraction of the monomers A and B is based on the total amount of the ethylenically unsaturated monomers that constitute the polymer.

EP 0693505—relates to curable liquid resins which are suitable for use as a coating composition capable of forming a film for use in for example inks or adhesives in the absence of a solvent.

U.S. Pat. No. 5,310,807 describes polymer dispersions of star polymers dispersed in an organic liquid; wherein the star polymer has a cross-linked core having attached thereto at least three macromolecular arms.

It has now been found that branched copolymers having a novel polymer architecture with a hydrophilic component can be prepared by an addition polymerisation method which have a variety of applications as a result of their advantageous properties. That is, the novel branched copolymers with a hydrophilic component can be prepared at high conversion rates, namely at 99% and greater than 99%, at a range of molecular weight values and give improved formulation properties such as a reduction in gelation when compared to a linear or “lightly branched” analogues.

Such branched addition copolymers find particular application where a range of molecular weight copolymers are required and which are either hydrophilic or comprise a component which is hydrophilic and where high solubility, or additional functionality is also required potentially with the advantage of high surface, substrate or co-ingredient interaction.

In addition, it has also now been found that the architecture of the branched addition copolymers show compactness of structure providing a high concentration of functionality not provided by linear materials.

Furthermore, the novel branched addition copolymers of this type which are either hydrophilic or comprise a component which is hydrophilic and with these properties find particular application is areas such as for example the petrochemical, construction, fuels or lubricants, electronics, agrochemical and pharmaceutical industries and may be used for example in coatings, inks, adhesives and sealants, construction, water-purification and water-softening, crystal growth inhibition, as sizing or wetting agents, freeze-point depressors, or in the home and personal care industries.

In the present invention the hydrophilic component comprises a combination of a residue of a hydrophilic monofunctional monomer with a solubility greater than 0.18% w/w in water at 20° C. and a residue of a hydrophilic multifunctional monomer a solubility greater than 0.18% w/w in water at 20° C. and/or a residue of a hydrophilic chain transfer agent with a solubility greater than 0.18% w/w in water at 20° C.

In addition, the hydrophilic component preferably comprises a hydrophilic moiety which can interact with aqueous media for example through charge or H-bonding. Hydrophilic moieties of this type preferably comprise but are not limited to acid, basic, amide, charged or H-bonding motif.

The copolymers of the present invention find particular application where copolymers with a hydrophilic residue are required. It has now been found that the incorporation of a hydrophilic residue derived form a combination of at least 1 mole % hydrophilic monofunctional monomer and hydrophilic multifunctional monomer and/or hydrophilic chain transfer agent as described above has a number of advantages, not least the added functionality this provides. Such hydrophilic functional groups derived form the hydrophilic residues comprise but are not limited to for example: carboxylic acids, alcohols and amines Copolymers possessing a hydrophilic component of this nature are able to demonstrate for example higher surface tension or adhesion and may therefore be utilised in for example coating formulations to superior effect compared with non-hydrophilically modified analogous polymers.

Additionally, the hydrophilic functional group may be post reacted to provide a modified ‘base’ polymer or a cross-linked material, where either the cross-linking reaction occurs between two mutually reactive polymers or via the use of a suitable reactive cross-linker molecule to connect two hydrophilically modified addition branched copolymers. This is particularly useful in the preparation of cross-linked resins, coatings, adhesives or membranes. Even at the incorporation of only 1 mole % of a combined hydrophilic component based on a combination of a residue of a hydrophilic monofunctional monomer with a solubility greater than 0.18% w/w in water at 20° C. and a residue of a hydrophilic multifunctional monomer with a solubility greater than 0.18% w/w in water at 20° C. and/or a residue of a hydrophilic chain transfer agent with a solubility greater than 0.18% w/w in water at 20° C. this increased functionality can be highly advantageous.

It has also now been found that the copolymers of the present invention can be utilised in a variety of fields and include applications for example, where copolymers are required which are either hydrophilic or comprise a component which is hydrophilic where high solubility, or additional functionality derived from the hydrophilic monomer residue the hydrophilic multifunctional monomers or the hydrophilic chain transfer agent is required, potentially with the advantage of high surface, substrate or co-ingredient interaction.

These properties may be required in such application areas as the petrochemical, construction, fuels or lubricants, electronics, agrochemical and pharmaceutical industries and used for example in coatings, inks, adhesives and sealants, construction, fuels or lubricants, electronics, water-purification and water-softening, crystal growth inhibition, as sizing or wetting agents, freeze-point depressors, or in the home and personal care industries.

Therefore according to a first aspect of the present invention there is provided a branched copolymer obtainable by an addition polymerisation process and comprising a hydrophilic component, said polymer comprising:
i) a residue of at least one monofunctional monomer comprising one polymerisable double bond per molecule and a molecular weight of less than 1000 Daltons;
ii) a residue of at least one multifunctional monomer comprising at least two polymerisable double bonds per molecule and a molecular weight of less than 1000 Daltons; and wherein the end termini of the copolymer chains comprise one or more of a residue of a chain transfer agent; an initiator or a terminal group derived from a termination reaction;
wherein;
   the molar ratio of the monofunctional monomer to multifunctional monomer is between 50:1 to 2.5:1 respectively; and
   wherein the hydrophilic component is comprised of at least 1 mole % of a combination of a monofunctional monomer, and a multifunctional monomer and/or a chain transfer agent when compared to the total content of monofunctional monomer which is/are comprised of hydrophilic component each with a solubility of 0.18 w/w % in water at 20° C.; and
   wherein the residue of the at least one monofunctional monomer with a molecular weight of less than 1000 Daltons is selected from the group comprising:
   vinyl acids, vinyl acid esters, vinyl aryl compounds, vinyl acid anhydrides, vinyl amides, vinyl ethers, vinyl amines, vinyl aryl amines, vinyl nitriles, vinyl ketones, and derivatives thereof;
   hydroxyl-containing monomers and monomers which can be post-reacted to form hydroxyl groups;
   acid-containing or acid functional monomers;
   zwitterionic monomers;
   quaternised amino monomers, oligomeric monomers; and corresponding allyl monomers of the aforesaid vinyl monomers.

The hydrophilic branched copolymer according to the present invention is prepared at a conversion rate of greater than or equal to 99%.

In the branched copolymers of the present invention between 1 to 100 mole %, of the at least one monofunctional monomer with a molecular weight of less than 1000 Daltons, at least one multifunctional monomer with a molecular weight of less than 1000 Daltons, and/or chain transfer agent is derived from a hydrophilic monofunctional monomer, hydrophilic multifunctional monomer and/or hydrophilic chain transfer agent respectively based on the total content of monofunctional monomer.

In the branched copolymers of the present invention between 10 to 100 mole %, of the at least one monofunctional monomer with a molecular weight of less than 1000 Daltons, at least one multifunctional monomer with a molecular weight of less than 1000 Daltons, and/or chain transfer agent is derived from a hydrophilic monofunctional monomer, hydrophilic multifunctional monomer and/or hydrophilic chain transfer agent based on the total content of monofunctional monomer.

In the branched copolymers of the present invention between 20 to 100 mole %, of the at least one monofunctional monomer with a molecular weight of less than 1000 Daltons, at least one multifunctional monomer with a molecular weight of less than 1000 Daltons, and/or chain transfer agent is derived from a hydrophilic monofunctional monomer, hydrophilic multifunctional monomer and/or hydrophilic chain transfer agent based on the total content of monofunctional monomer.

Also in the branched copolymers of the present invention the molar concentration of multifunctional monomer relative to the amount of monofunctional monomer is greater than or equal to (≧) 2. Preferably, the molar concentration of multifunctional monomer relative to the amount of monofunctional monomer is 2 to 50. More preferably the molar concentration of multifunctional monomer relative to the amount of monofunctional monomer is 2 to 40. Most preferably the molar concentration of multifunctional monomer relative to the amount of monofunctional monomer is 2 to 30. However, the molar concentration of multifunctional monomer relative to the amount of monofunctional monomer is especially 2 to 15.

Also in connection with the branched copolymers of the present invention the multifunctional monomer comprises a residue of a multifunctional monomer selected from the group comprising di- or multivinyl esters, di- or multivinyl amides, di- or multivinyl aryl compounds and di- or multivinyl alk/aryl ethers.

Most preferably, the multifunctional monomer comprises a residue of a multifunctional monomer selected from the group comprising a multifunctional monomer containing two or more polymerisable groups where the total weight average molecular weight of the molecule is less than 1000 Da. Where the multifunctional monomer is hydrophilic in nature the molecule has solubility in water of greater than 0.18% w/w at 20° C. Preferred hydrophilic multifunctional monomers include ethyleneglycol di(methacrylate), propylene glycol di(meth)acrylate, and poly(ethyleneglycol)di(meth)acrylate, poly(propyleneglycol)di(meth)-acrylate.

When the branched copolymer of the present invention comprises a chain transfer agent, the residue of the chain transfer agent comprises between 0 to 50 mole %, of the copolymer. Preferably the residue of the chain transfer agent comprises between 0 to 40 mole %, of the copolymer. Most preferably however the residue of the chain transfer agent comprises between 0.05 to 30 mole %, of the copolymer.

The chain transfer agent is selected from the group comprising: monofunctional and multifunctional thiols and alkyl halides and other compounds known to be active in free radical chain transfer processes such as 2,4-diphenyl-4-methyl-1-pentene.

Suitable thiols include but are not limited to: C2-C18 alkyl thiols such as dodecane thiol. Thiol-containing oligomers may also be used such as oligo(cysteine) or an oligomer which has been post-functionalised to give a thiol group(s), such as oligoethylene glycolyl(di)thio glycollate, thiopropionic acid and esters thereof such as butyl-3-mercaptopropionate and octyl-3-mercaptopropionate, thiolactic acid. Preferred thiols include linear or branched alkylthiols such as dodecyl mercaptan, thio alcohols such as thioethanol, thio alky esters such as octyl-3-mercaptopropionate and thio acids such as thio lactic acid. Xanthates, dithioesters, and dithiocarbonates may also be used, such as cumyl phenyldithioacetate.

More preferred chain transfer agents comprise: thiolactic acid, thioglycolic acid, thioglycerol, thioethanol, cysteine and cysteamine.

In addition, the chain transfer agent may comprise a compound which reduces the molecular weight of a copolymer during a free radical polymerisation reaction. It is also preferred that the chain transfer agent has a molecular weight of 1000 Daltons or less.

When the branched copolymer according to the present invention comprises an initiator, the residue of the initiator comprises between 0 to 15% w/w of the copolymer based on the total weight of the monomers. More preferably, the residue of the initiator comprises between 0.01 to 10% w/w, of the copolymer based on the total weight of the monomers.

The initiator is preferably selected from the group comprising: persulfates, redox initiators, peroxides, dialkylperoxides, peroxybenzoates and benzyl ketones. Most preferably dialkylperoxides and peroxybenzoates.

More preferred initiators comprise: dialkylperoxides, alkyl/aryl hydroperoxides and peroxybenzoates with a one hour half-life temperature above 82° C.

The weight average molecular weight (Mw) of the copolymer is preferably between 5 and 1500 kDa. Most preferably however, the weight average molecular weight (Mw) of the copolymer according to the present invention is in the range 10 to 1500. However, the weight average molecular weight (Mw) of the copolymer may be greater than or equal to 20 kDa.

Furthermore, in the branched copolymers according to the present invention, the residue of at least one monofunctional monomer with a molecular weight of less than 1000 Daltons is selected from the group comprising (meth)acrylates, styrenics, (meth)acrylamides, N-vinyl alkamides, vinyl alkylates.

Also, in the branched copolymers according to the present invention, the hydrophilic monomer with a solubility of 0.18 w/w % in water at 20° C. forming the hydrophilic component of the copolymer is selected from the group comprising; (meth)acrylates, styrenics, (meth)acrylamides, N-vinyl alkamides, vinyl ester, vinyl amides and vinyl alkylates

More preferably, the hydrophilic monomer with a solubility of 0.18 w/w % in water forming the hydrophilic component of the copolymer is selected from the group comprising; (meth)acrylates, (meth)acrylamide and styrenics.

The preferred copolymers according to the present invention comprise (meth)acrylate, (meth)acrylamide, or styrenic-based co-polymers containing a hydrophilic moiety such as an acid, basic, ether, amide or ester group which interact with water through charge or H-bonding.

Preferred hydrophilic monofunctional monomers include: amide-containing monomers such as (meth)acrylamide, [3-((meth)acrylamido)propyl]trimethyl ammonium chloride, 3-dimethylamino)propyl(meth)acrylamide, 3-[N-(3-(meth)acrylamidopropyl)-N,N-dimethyl]aminopropane sulfonate, methyl(meth)acrylamidoglycolate methyl ether and N-isopropyl(meth)acrylamide; (meth)acrylic acid and derivatives thereof such as (meth)acrylic acid, (meth)acryloyl chloride (or any halide), functionalised oligomeric monomers such as monomethoxy oligo(ethyleneglycol)mono(meth)acrylate, monomethoxy oligo(propyleneglycol)mono(meth)acrylate, monohydroxy oligo(ethyleneglycol)mono(meth)acrylate, monohydroxy oligo(propyleneglycol)mono(meth)acrylate, glycerol mono(meth)acrylate and sugar mono(meth)acrylates such as glucose mono(meth)acrylate; vinyl amines such as aminoethyl(meth)acrylate, dimethylaminoethyl(meth)acrylate, diethylaminoethyl(meth)acrylate, diisopropylaminoethyl(meth)acrylate, mono-t-butylaminoethyl(meth)acrylate, morpholinoethyl(meth)acrylate, vinyl aryl amines such as vinyl aniline, vinyl pyridine, N-vinyl carbazole and monomers which can be post-reacted to form amine groups, such as vinyl formamide; vinyl aryl monomers such as styrene sulfonic acid and vinyl benzoic acid; Vinyl hydroxyl monomers such as hydroxyethyl(meth)acrylate, hydroxy propyl(meth)acrylate, glycerol mono(meth)acrylate and monomers which can be post-functionalised into hydroxyl groups such as vinyl acetate, acetoxystyrene and glycidyl (meth)acrylate; acid-containing monomers such as (meth)acrylic acid, styrene sulfonic acid, vinyl phosphonic acid, vinyl benzoic acid, maleic acid, fumaric acid, itaconic acid, 2-(meth)acrylamido 2-ethyl propanesulfonic acid and mono-2-((meth)acryloyloxy)ethyl succinate; and their respective onium salts. zwitterionic monomers such as (meth)acryloyl oxyethylphosphoryl choline and betaine-containing monomers, such as [2-((meth)acryloyloxy)ethyl]dimethyl-(3-sulfopropyl)ammonium hydroxide; and quaternised amino monomers such as (meth)acryloyloxyethyltrimethyl ammonium chloride.

Preferred hydrophilic monofunctional monomers include: amide-containing monomers such as (meth)acrylamide, [3-((meth)acrylamido)propyl]trimethyl ammonium chloride, methyl(meth)acrylamidoglycolate methyl ether and N-isopropyl(meth)acrylamide; (meth)acrylic acid and derivatives thereof such as (meth)acrylic acid, (meth)acryloyl chloride (or any halide), functionalised oligomeric monomers such as monomethoxy oligo(ethyleneglycol)mono(meth)acrylate, monomethoxy oligo(propyleneglycol)mono(meth)acrylate, monohydroxy oligo(ethyleneglycol)mono(meth)acrylate, monohydroxy oligo(propyleneglycol)mono(meth)acrylate, vinyl amines such as aminoethyl(meth)acrylate, dimethylaminoethyl(meth)acrylate, diethylaminoethyl(meth)acrylate, vinyl aryl amines such as vinyl pyridine, and monomers which can be post-reacted to form amine groups, such as vinyl formamide; vinyl aryl monomers such as styrene sulfonic acid and vinyl benzoic acid; Vinyl hydroxyl monomers such as hydroxyethyl(meth)acrylate, hydroxy propyl(meth)acrylate, and monomers which can be post-functionalised into hydroxyl groups such as vinyl acetate, acetoxystyrene and glycidyl(meth)acrylate; acid-containing monomers such as (meth)acrylic acid, styrene sulfonic acid, vinyl phosphonic acid, vinyl benzoic acid, maleic acid, fumaric acid, itaconic acid, 2-(meth)acrylamido 2-ethyl propanesulfonic acid and mono-2-((meth)acryloyloxy)ethyl succinate; and their respective onium salts, zwitterionic monomers such as and betaine-containing monomers, such as [2-((meth)acryloyloxy)ethyl]dimethyl-(3-sulfopropyl)ammonium hydroxide; and quaternised amino monomers such as (meth)acryloyloxyethyltrimethyl ammonium chloride.

According to a second aspect of the present invention there is provided a method of preparing a branched copolymer with a hydrophilic component according to any one of the preceding claims by an addition process which comprises forming an admixture of:
a) at least one monofunctional monomer;
b) at least 2 mole % of a multifunctional monomer relative to the number of moles of monofunctional monomer;
c) a chain transfer agent; and/or
(d) an initiator; all as previously defined in relation to the first aspect of the present invention and subsequently reacting said mixture to form a branched copolymer by a solution process and wherein the hydrophilic branched copolymer according to the present invention is prepared at a conversion rate of greater than or equal to 99%.

A solution process refers to a process where following the polymerisation reaction a solution of polymer in a liquid is obtained. An example of this would be where a solvating liquid is used to dissolve the constituents of the polymerisation, monofunctional monomer(s), multifunctional monomer(s), chain transfer agent(s) and initiator(s), and following polymerisation a solution of polymer is obtained.

A further example would be where the monomer is dispersed during the polymerisation process and upon polymerisation the polymer is obtained as a low viscosity latex solution of polymer in solvent.

According to a third aspect of the present invention there is provided a polymer dispersion or solution of the branched copolymer according to the present invention wherein the copolymer is dissolved or dispersed in an aqueous or non-aqueous solvent or emulsion.

Therefore there is also provided in accordance with the present invention a composition comprising:
i) a branched copolymer with a hydrophilic component from a residue of a hydrophilic monofunctional monomer and/or a multifunctional monomer and/or a chain transfer agent according to a first aspect of the present invention; and
ii) an aqueous or non-aqueous solution or emulsion wherein the branched copolymer is dispersed or dissolved in the solution or emulsion.

When the composition comprises an aqueous solution or aqueous emulsion, the aqueous solution or aqueous emulsion comprises;

water, a salt solution at varying concentrations, an aqueous co-solvent, an aqueous emulsion or an aqueous solution at pH 0 to 14, at temperatures varying between minus (−) 20° C. to 140° C.

Finally, according to a fourth aspect of the present invention there is provided the use of a branched copolymer with a hydrophilic component according to the first or third aspects of the present invention in the petrochemical, agrochemical and pharmaceutical industries and for coatings, inks, adhesives and sealants, construction, fuels or lubricants, electronics, water-purification and water-softening, crystal growth inhibition, sizing or wetting agent, freeze-point depressor, or in the home and personal care industries.

### DETAILED DESCRIPTION OF THE INVENTION

### Definitions

The following definitions pertain to chemical structures, molecular segments and substituents:

The term “alkyl” as used herein refers to a branched or unbranched saturated hydrocarbon group which may contain from 1 to 12 carbon atoms, such as methyl, ethyl, n-propyl, isopropyl, n-butyl, isobutyl, t-butyl, octyl, decyl etc. More preferably, an alkyl group contains from 1 to 6, preferably 1 to 4 carbon atoms. Methyl, ethyl, propyl and butyl groups are especially preferred. “Substituted alkyl” refers to alkyl substituted with one or more substituent groups. Preferably, alkyl and substituted alkyl groups are unbranched.

Typical substituent groups include, for example: halogen atoms, nitro, cyano, hydroxyl, cycloalkyl, alkyl, alkenyl, haloalkyl, alkoxy, haloalkoxy, amino, alkylamino, dialkylamino, formyl, alkoxycarbonyl, carboxyl, alkanoyl, alkylthio, alkylsulfinyl, alkylsulfonyl, alkylsulfonato, arylsulfinyl, arylsulfonyl, arylsulfonato, phosphinyl, phosphonyl, carbamoyl, amido, alkylamido, aryl, aralkyl and quaternary ammonium groups, such as betaine groups. Of these substituent groups, halogen atoms, cyano, hydroxyl, alkyl, haloalkyl, alkoxy, haloalkoxy, amino, carboxyl, amido and quaternary ammonium groups, such as betaine groups, are particularly preferred. When any of the foregoing substituents represents or contains an alkyl or alkenyl substituent group, this may be linear or branched and may contain up to 12, preferably up to 6, and especially up to 4, carbon atoms. A cycloalkyl group may contain from 3 to 8, preferably from 3 to 6, carbon atoms. An aryl group or moiety may contain from 6 to 10 carbon atoms, phenyl groups being especially preferred. A halogen atom may be a fluorine, chlorine, bromine or iodine atom and any halo group one may be one that contains a halo moiety, such as a haloalkyl group, may thus contain any one or more of these halogen atoms.

Terms such as “(meth)acrylic acid” embrace both methacrylic acid and acrylic acid. Analogous terms should be construed similarly.

Terms such as “alk/aryl” embrace alkyl, alkaryl, aralkyl (for example, benzyl) and aryl groups and moieties.

Molar percentages are based on the total monofunctional monomer content.

Molecular weights of monomers and polymers are expressed as weight average molecular weights, except where otherwise specified.

### The Copolymers

The branched copolymers of the present invention with a hydrophilic component derived from the residue of a hydrophilic monofunctional monomer and/or a multifunctional monomer and/or a chain transfer agent are branched addition polymers and include statistical, gradient and alternating branched copolymers.

More specifically, the polymer, that is a copolymer structure comprises:

a residue of a chain transfer agent and/or an initiator;

a residue of at least one monofunctional monomer having one polymerisable double bond per molecule and a molecular weight of less than 1000 Daltons;

a residue of a multifunctional monomer having at least two polymerisable double bonds per molecule and a molecular weight of less than 1000 Daltons;

a residue of a terminal group derived from a termination reaction, wherein the end termini of the copolymer chains comprise one or more of a residue of a chain transfer agent; an initiator or a terminal group derived from a termination reaction; and

wherein, the molar ratio of multifunctional monomers to monofunctional monomers is greater than or equal to 1:50 respectively; and
wherein the copolymer comprises a hydrophilic component and wherein the hydrophilic component is comprised of at least 1 mole % of a combination of a monofunctional monomer, and a multifunctional monomer and/or a chain transfer agent when compared to the total content of monofunctional monomer which is/are comprised of hydrophilic component each with a solubility of 0.18 w/w % in water at 20° C.; and

wherein the residue of the at least one monofunctional monomer with a molecular weight of less than 1000 Daltons is selected from the group comprising:
vinyl acids, vinyl aryl compounds, vinyl acid anhydrides, vinyl amides, vinyl ethers, vinyl amines, vinyl aryl amines, vinyl nitriles, vinyl ketones, and derivatives thereof;
hydroxyl-containing monomers and monomers which can be post-reacted to form hydroxyl groups;
acid-containing or acid functional monomers;
zwitterionic monomers;
amide functional monomers;
ether functional monomers;
quaternised amino monomers, oligomeric monomers; and corresponding allyl monomers of the aforesaid vinyl monomers.

An advantage of the present invention is that the branched copolymers may be and are preferably prepared at a conversion rate of greater than or equal to 99%.

The copolymer may also contain unreacted vinyl groups from the multifunctional monomer.

The monofunctional monomer may comprise any carbon-carbon unsaturated compound which can be polymerised by an addition polymerisation mechanism, for example, vinyl and allyl compounds. The monofunctional monomer may be selected from monomers which are hydrophilic, hydrophobic, amphiphilic, anionic, cationic, neutral or zwitterionic in nature.

The monofunctional monomer may be selected from but not limited to monomers such as: vinyl acids, vinyl acid esters, vinyl aryl compounds, vinyl acid anhydrides, vinyl amides, vinyl ethers, vinyl amines, vinyl aryl amines, vinyl nitriles, vinyl ketones, and derivatives of the aforementioned compounds as well as corresponding allyl variants thereof. Other suitable monofunctional monomers include: hydroxyl-containing monomers and monomers which can be post-reacted to form hydroxyl groups, acid-containing or acid functional monomers, zwitterionic monomers and quaternised amino monomers. Oligomeric or oligo-functionalised monomers may also be used, especially oligomeric (meth)acrylic acid esters such as mono(alk/aryl) (meth)acrylic acid esters of oligo(alkyleneglycol) or oligo(dimethylsiloxane) or any other mono-vinyl or allyl adduct of a low molecular weight oligomer. Mixtures of more than one monomer may also be used to give statistical, gradient or alternating copolymers. The monofunctional monomer most preferably comprises a molecular weight of less than 1,000 Daltons. Thus the monofunctional monomer is represented by a residue of a monofunctional monomer as described above.

Vinyl acids and derivatives thereof include: (meth)acrylic acid and acid halides thereof such as (meth)acryloyl chloride. Vinyl acid esters and derivatives thereof include C₁₋₂₀ alkyl(meth)acrylates (linear & branched) such as methyl(meth)acrylate, stearyl (meth)acrylate and 2-ethyl hexyl(meth)acrylate, aryl(meth)acrylates such as benzyl (meth)acrylate, tri(alkyloxy)silylalkyl(meth)acrylates such as trimethoxysilylpropyl(meth)acrylate and activated esters of (meth)acrylic acid such as N-hydroxysuccinamido (meth)acrylate.

Vinyl acid anhydrides and derivatives thereof include: maleic anhydride. Vinyl amides and derivatives thereof include: (meth)acrylamide, N-vinyl formamide, (meth)acrylamidopropyl trimethyl ammonium chloride, [3-((meth)acrylamido)propyl]dimethyl ammonium chloride, [3-(meth)acrylamidopropyl)-N,N-dimethyl]aminopropane sulfonate, methyl(meth)acrylamidoglycolate methyl ether and N-isopropyl(meth)acrylamide. Vinyl ethers and derivatives thereof include: methyl vinyl ether. Vinyl amines and derivatives thereof include: dimethylaminoethyl(meth)acrylate, diethylaminoethyl(meth)acrylate, diisopropylaminoethyl(meth)acrylate, mono-t-butylaminoethyl(meth)acrylate, morpholinoethyl(meth)acrylate and monomers which can be post-reacted to form amine groups, such as vinyl formamide. Vinyl aryl amines and derivatives thereof include vinyl aniline, vinyl pyridine and N-vinyl carbazole. Vinyl nitriles and derivatives thereof include (meth)acrylonitrile. Vinyl ketones and derivatives thereof include acreolin.

Hydroxyl-containing monomers include: vinyl hydroxyl monomers such as hydroxyethyl(meth)acrylate, hydroxy propyl(meth)acrylate, glycerol mono(meth)acrylate and sugar mono(meth)acrylates such as glucose mono(meth)acrylate. Monomers which can be post-reacted to form hydroxyl groups include: vinyl acetate, acetoxystyrene and glycidyl (meth)acrylate. Acid-containing or acid functional monomers include: (meth)acrylic acid, styrene sulfonic acid, vinyl phosphonic acid, vinyl benzoic acid, maleic acid, fumaric acid, itaconic acid, 2-(meth)acrylamido 2-ethyl propanesulfonic acid, mono-2-((meth)acryloyloxy)ethyl succinate and ammonium sulfatoethyl(meth)acrylate. Zwitterionic monomers include: (meth)acryloyl oxyethylphosphoryl choline and betaines, such as [2-((meth)acryloyloxy) ethyl]dimethyl-(3-sulfopropyl)ammonium hydroxide. Quaternised amino monomers include: (meth)acryloyloxyethyltri-(alk/aryl)ammonium halides such as (meth)acryloyloxyethyltrimethyl ammonium chloride.

Oligomeric monomers include: oligomeric (meth)acrylic acid esters such as mono(alk/aryl)oxyoligoalkyleneoxide(meth)acrylates and mono(alk/aryl)oxyoligodimethyl-siloxane(meth)acrylates. These esters include: monomethoxy oligo(ethyleneglycol)mono(meth)acrylate, monomethoxy oligo(propyleneglycol)mono(meth)acrylate, monohydroxy oligo(ethyleneglycol)mono(meth)acrylate and monohydroxy oligo(propyleneglycol)mono(meth)acrylate. Further examples include: vinyl or allyl esters, amides or ethers of pre-formed oligomers formed via ring-opening polymerisation such as oligo(caprolactam) or oligo(caprolactone), or oligomers formed via a living polymerisation technique such as oligo(1,4-butadiene).

The corresponding allyl monomers to those listed above can also be used where appropriate.

It is essential that the copolymer of the present invention comprises a hydrophilic component comprised of at least 1 mole % of a hydrophilic component derived from a combination of at least 1 mole % hydrophilic monofunctional monomer and hydrophilic multifunctional monomer and/or hydrophilic chain transfer agent in order to achieve the desired level of functionality required for the applications of these materials.

Ideally, 1 to 99 mole % of the monofunctional monomer and multifunctional monomer and/or hydrophilic chain transfer agent is derived from hydrophilic residues. Preferably at least 10 mole % and, more preferably, at least 20 mole %, of the hydrophilic monofunctional monomer, multifunctional monomer and hydrophilic chain transfer agent is derived from hydrophilic residues. Most preferably 30% of the hydrophilic monofunctional monomer, m hydrophilic multifunctional monomer and hydrophilic chain transfer agent is derived from a hydrophilic residues. Molar percentages are based on the total monofunctional monomer content.

The final copolymer with a hydrophilic component may be water soluble or dispersible and soluble or dispersible within an aqueous environment.

The aqueous environment may be comprised of water at varying salt concentrations, pH levels, temperatures and with or without a co-solvent wherein the water miscible co-solvents are selected from the group comprising: lower alcohols, including but not limited to: methanol, ethanol, propanol, isopropanol, n-butanol, iso- or tert-butanol; ketones or aldehydes including acetone; esters including ethyl acetate; amides such as N—N′-dimethyl acetamide or N—N′-dimethyl formamide; sulfoxides such as dimethylsulfoxide or mixtures thereof.

The aqueous medium may further comprise an aqueous emulsion, either oil-in-water, or water-in-oil where the branched addition copolymer with a hydrophilic component as described above is dissolved or dispersed in the aqueous phase. Such emulsions may comprise hydrophobic oils including but not limited to: hydrocarbons, higher alcohols, cosmetic oils, natural oils and the like dispersed with a surface active agent wherein the polymer is present during the emulsification step or is added to the pre-formed emulsion.

Suitable hydrophilic or water-soluble monofunctional monomers are soluble in water across a pH range of 0 to 14 at a level greater than 0.18% w/w in water at 20° C. The monomers preferably contain a water solubilising group such as a H-bonding moiety or a permanent or transient anionic or cationic charge, or both.

Table 1 illustrates a non-exhaustive illustrative list of various monomers with a solubility in water at greater than 0.18% w/w at 20° C. and a hydrophilic functional group such as an acid, amine (in neutral of ionic state), hydroxyl, amide, ester, ether and epoxy.

TABLE 1
Water solubilities of some hydrophilic monomer examples

| Monomer | Solubility in water w/w at 20° C. |
|---|---|
| Acrylic acid | Soluble |
| 2-Hydroxyethyl acrylate | soluble |
| 2-Hydroxyethyl methacrylate | 3.00 |
| 2-Hydroxypropyl acrylate | Soluble |
| 2-Hydroxypropyl methacrylate | 2.17 |
| Methacrylic acid | Soluble |
| 4-vinyl pyridine | 2.91 |

### Hydrophilic Multifunctional Monomers:

Ethyleneglycol di(methacrylate), propyleneglycol di(meth)acrylate, poly(ethyleneglycol)di(meth)acrylate, poly(propyleneglycol)di(meth)acrylate,

### Hydrophilic Chain Transfer Agents

Thiolactic acid, thioglycolic acid, thioglycerol, thioethanol, cysteine and cysteamine

Examples of hydrophilic monofunctional monomers include but are not limited to: vinyl amides and derivatives thereof, (meth)acrylic acid and derivatives thereof such as acid halides, activated esters of (meth)acrylic acid, vinyl amines, vinyl aryl monomers, hydroxyl-containing monomers or monomers which can be post-reacted to form alcohols, acid-containing or acid functional monomers, aromatic amine monomers, vinyl ethers, vinyl nitriles, vinyl ketones, zwitterionic monomers and quaternised amino monomers as described above.

More preferred hydrophilic monomers include: amide-containing monomers such as (meth)acrylamide, [3-((meth)acrylamido)propyl]trimethyl ammonium chloride, 3-(dimethylamino)propyl(meth)acrylamide, 3-[N-(3-(meth)acrylamidopropyl)-N,N-dimethyl]aminopropane sulfonate, methyl(meth)acrylamidoglycolate methyl ether and N-isopropyl(meth)acrylamide; (meth)acrylic acid and derivatives thereof such as (meth)acrylic acid, (meth)acryloyl chloride (or any halide), functionalised oligomeric monomers such as monomethoxy oligo(ethyleneglycol)mono(meth)acrylate, monomethoxy oligo(propyleneglycol)mono(meth)acrylate, monohydroxy oligo(ethyleneglycol)mono(meth)acrylate, monohydroxy oligo(propyleneglycol)mono(meth)acrylate, glycerol mono(meth)acrylate and sugar mono(meth)acrylates such as glucose mono(meth)acrylate; vinyl amines such as aminoethyl(meth)acrylate, dimethylaminoethyl(meth)acrylate, diethylaminoethyl(meth)acrylate, diisopropylaminoethyl(meth)acrylate, mono-t-butylaminoethyl(meth)acrylate, morpholinoethyl(meth)acrylate, vinyl aryl amines such as vinyl aniline, vinyl pyridine, N-vinyl carbazole and monomers which can be post-reacted to form amine groups, such as vinyl formamide; vinyl aryl monomers such as styrene sulfonic acid and vinyl benzoic acid;

Vinyl hydroxyl monomers such as hydroxyethyl(meth)acrylate, hydroxy propyl (meth)acrylate, glycerol mono(meth)acrylate and monomers which can be post-functionalised into hydroxyl groups such as vinyl acetate, acetoxystyrene and glycidyl(meth)acrylate; acid-containing monomers such as (meth)acrylic acid, styrene sulfonic acid, vinyl phosphonic acid, vinyl benzoic acid, maleic acid, fumaric acid, itaconic acid, 2-(meth)acrylamido 2-ethyl propanesulfonic acid and mono-2-((meth)acryloyloxy)ethyl succinate; and their respective onium salts.

zwitterionic monomers such as (meth)acryloyl oxyethylphosphoryl choline and betaine-containing monomers, such as [2-((meth)acryloyloxy)ethyl]dimethyl-(3-sulfopropyl)ammonium hydroxide; and quaternised amino monomers such as (meth)acryloyloxyethyltrimethyl ammonium chloride.

The corresponding allyl monomer, where applicable, can also be used in each case.

Examples of water-insoluble monomers include extremely hydrophobic materials such as styrene (water solubility 0.02% w/w) and 2-ethyl hexyl acrylate (0.01% w/w).

Hydrophobic monomers include: vinyl aryl compounds such as styrene and vinylbenzyl chloride; (meth)acrylic acid esters such as mono-t-butylaminoethyl(meth)acrylate, C1-20 alkyl(meth)acrylates (linear & branched), aryl(meth)acrylates, such as benzyl methacrylate; ligomeric (meth)acrylic acid esters such as mono(alk/aryl)oxyoligo[dimethylsiloxane(meth)acrylate] and tri(alkyloxy)silylalkyl(meth)acrylates such as trimethoxysilylpropyl(meth)acrylate.

Functional monomers, that is, monomers with reactive pendant groups which can be post or pre-modified with another moiety can also be used such as glycidyl(meth)acrylate, trimethoxysilylpropyl(meth)acrylate, (meth)acryloyl chloride, maleic anhydride, hydroxyalkyl(meth)acrylates, (meth)acrylic acid, vinylbenzyl chloride, activated esters of (meth)acrylic acid such as N-hydroxysuccinamido(meth)acrylate and acetoxystyrene.

The multifunctional monomer may comprise a molecule containing at least two vinyl groups which may be polymerised via addition polymerisation. The molecule may be hydrophilic, hydrophobic, amphiphilic, neutral, cationic, zwitterionic or oligomeric. Such molecules are often known as crosslinking agents in the art and may be prepared by reacting any di or multifunctional molecule with a suitably reactive monomer. The multifunctional monomer comprises at least two polymerisable double bonds per molecule also has a molecular weight less than 1,000 Daltons. Examples include di- or multivinyl esters, di- or multivinyl amides, di- or multivinyl aryl compounds and di- or multivinyl alk/aryl ethers. Typically, in the case of oligomeric or multifunctional branching agents, a linking reaction is used to attach a polymerisable moiety to a di- or multifunctional oligomer or a di- or multifunctional group. The brancher may itself have more than one branching point, such as T-shaped divinylic oligomers. In some cases, more than one multifunctional monomer may be used.

The corresponding allyl monomers to those listed above can also be used where appropriate.

Thus, the multifunctional monomer is a residue of a multifunctional monomer as described above.

Preferred hydrophilic multifunctional monomers include but are not limited to: ethyleneglycol di(methacrylate), propyleneglycol di(meth)acrylate, poly(ethyleneglycol)di(meth)acrylate, poly(propyleneglycol)di(meth)acrylate,

Thus, the multifunctional monomer is a residue of a multifunctional monomer as described above.

Preferred hydrophobic multifunctional monomers, with a solubility in water at 20° C. is less than 0.18% w/.w, include but are not limited to: divinyl benzene; 1,3-butylenedi(meth)acrylate; 1,6-hexanediol di(meth)acrylate, silicone-containing divinyl esters or amides such as (meth)acryloxypropyl-terminated oligo(dimethylsiloxane). Further examples include vinyl or allyl esters, amides or ethers of pre-formed oligomers formed via ring-opening polymerisation such as oligo(caprolactam) or oligo(caprolactone), or oligomers formed via a living polymerisation technique such as oligo(1,4-butadiene).

The ratio between the monofunctional monomer and the multifunctional monomer is preferably in the range 50:1 and 2.5:1. It is preferred that the molar ratios have a value of at least 50:1. Preferably a range of 40:1. More preferably 20:1 and particularly 10:1. It is especially preferred that the range is 7:1 in order to give the benefits associated with a branched polymer over a high molecular weight macromolecule.

It is also preferred that the weight average molecular weight (Mw) of the polymer is between 10 to 1500 k Da. The weight average molecular weight (Mw) of the polymer may also be greater than or equal to 20 kDa. In addition, the weight average molecular weight (Mw) of the polymer is greater than or equal to 25 kDa. It can also be that the weight average molecular weight (Mw) of the polymer is equal to 30 kDa.

The copolymer may be prepared by an addition polymerisation method, preferably either by a conventional free-radical polymerisation technique using a chain transfer agent or via a living radical polymerisation technique.

The chain transfer agent is a molecule that is known to reduce molecular weight during a free-radical polymerisation via a chain transfer mechanism. These agents may be any thiol-containing molecule and can be either monofunctional or polyfunctional. The agent may be hydrophilic, hydrophobic, amphiphilic, anionic, cationic, neutral or zwitterionic. The molecule can also be an oligomer containing a thiol moiety. Suitable thiols include but are not limited to C₂-C₁₈ alkyl thiols such as dodecane thiol. Thiol-containing oligomers may also be used such as oligo(cysteine) or an oligomer which has been post-functionalised to give a thiol group(s), such as oligoethylene glycolyl(di)thio glycollate, and thiopropionic acid esters such as butyl-3-mercaptopropionate and octyl-3-mercaptopropionate. Additionally other compounds known to be active in free radical chain transfer processes such as 2,4-diphenyl-4-methyl-1-pentene can be used. Xanthates, dithioesters, and dithiocarbonates may also be used, such as cumyl phenyldithioacetate. Alternative chain transfer agents may be any species known to limit the molecular weight in a free-radical addition polymerisation including alkyl halides and transition metal salts or complexes. More than one chain transfer agent may be used in combination. Ideally, the chain transfer agent has a molecular weight of 1000 Daltons or less. More preferably less than 1000 Daltons.

Preferred hydrophilic chain transfer agents include: thiolactic acid, thioglycolic acid, thioglycerol, thioethanol, cysteine and cysteamine.

The residue of the chain transfer agent may comprise 0 to 50 mole %, preferably 0 to 40 mole % and especially 0.05 to 30 mole %, of the copolymer (based on the number of moles of monofunctional monomer).

In the case of free-radical polymerisation, the initiator is a free-radical initiator and can be any molecule known to initiate free-radical polymerisation such as, persulfates, redox initiators, organic peroxides, organic peroxyacids and aromatic ketones. These may be activated via thermal, photolytic or chemical means. Examples of these include but are not limited to, benzoyl peroxide, di-t-butyl peroxide, t-butyl peroxybenzoate, cumylperoxide, 1-hydroxycyclohexyl phenyl ketone, hydrogen peroxide/ascorbic acid. Iniferters such as benzyl-N,N-diethyldithiocarbamate can also be used. In some cases, more than one initiator may be used.

Preferably, the residue of the initiator in a free-radical polymerisation comprises 0 to 15% w/w, preferably 0.01 to 12% w/w and especially 0.01 to 10% w/w, of the copolymer based on the total weight of the monomers.

The use of a chain transfer agent and an initiator is preferred. However, some molecules can perform both functions.

Additionally the polymer structure contains a terminal group derived from a termination reaction. During conventional radical polymerisation, some inherent and unavoidable termination reactions occur. Common termination reactions between free-radicals are typically bimolecular combination and disproportionation reactions which vary depending on the monomer structure and result in the annihilation of two radicals. Disproportionation reactions are thought to be the most common, especially for the polymerisation of (meth)acrylates, and involve two dead primary chains, one with a hydrogen terminus and the other with a carbon-carbon double bond. When the termination reaction is a chain transfer reaction the terminal unit is an easily abstractable atom, commonly hydrogen. Thus, for instance, when the chain transfer agent is a thiol, the terminal unit can be a hydrogen atom.

### Synthesis of the Copolymers

As mentioned above, the copolymers of the invention are prepared by an addition polymerisation method. This process is typically a conventional free-radical polymerisation process. Conventional free-radical polymerisation is particularly preferred.

To produce a branched polymer by a conventional free-radical polymerisation process, a monofunctional monomer is polymerised with a multifunctional monomer or branching agent in the presence of a chain transfer agent and free-radical initiator.

The polymerisations may proceed via solution, bulk, suspension, dispersion and emulsion procedures.

Most preferably the present invention proceeds via a solution procedure whereby the polymer is obtained as a solution in a solvent following the polymerisation reaction.

Thus, the invention also provides a method of preparing a branched copolymer with a hydrophilic component derived from the residue of a hydrophilic monofunctional monomer and a hydrophilic multifunctional monomer and/or a chain transfer agent as defined above in relation to a first aspect of the present invention by an addition process which comprises forming an admixture of:
(a) at least one monofunctional monomer;
(b) at least 2 mole % of a multifunctional monomer relative to the number of moles of monofunctional monomer;
(c) a chain transfer agent; and/or
(d) an initiator; all as previously defined in relation to the first aspect of the invention and subsequently reacting said mixture to form a branched copolymer.
The method of the present invention is very successful and be achieved at greater than or equal to 99% conversion.

### Compositions

The branched addition copolymers comprising a hydrophilic component according to the present invention find particular applications in aqueous media as a result of their potential high molecular weight, high solubility and functionality this lend them to multiple applications. Where a residue of a hydrophilic component is present in the resulting branched copolymer the increased functionality can improve surface adhesion and is available for further reactive steps post polymerisation such as crosslinking of post-functionalisation.

The architecture of the polymers can also have an effect on the pKa of polyacids or bases where the polymer is composed of mostly basic or acidic moieties due to the architectural arrangement. Thus, the copolymers of the invention may be used in a variety of applications. However, the copolymers of the invention find particular application where one or more a branched copolymers with a hydrophilic component are required in a formulation where the polymers have a solubility or dispersibility of at least 0.1 g per litre. Preferably the polymers have a solubility or dispersibility of at least 0.2 g per litre. More preferably the polymers have a solubility or dispersibility of at least 0.5 g per litre, particularly 1 g per litre. Especially the polymers have a solubility or dispersibility of at least 2 g per litre.

The present invention will now be explained in more detail by reference to the following non-limiting examples:—

### Examples

In the following examples, copolymers are described using the following nomenclature:—

(Monofunctional Monomer G)g (Monofunctional Monomer J)j (Multifunctional L)1 (Chain Transfer Agent D)d

wherein the values in subscript are the molar ratios of each constituent normalised to give the monofunctional monomer values as 100, that is, g plus j is equal to 100 (g+j=100). The degree of branching or branching level is denoted by 1 and d refers to the molar ratio of the chain transfer agent.

For example:—

Methacrylic acid100 Divinyl benzene15 Dodecane thiol15 would describe a polymer containing methacrylic acid:divinyl benzene:dodecane thiol at a molar ratio of 100:15:15.

### Preparation of Branched Addition Polymers Via a Solution Procedure:

The examples described were prepared via a solution polymerisation procedure. In a typical reaction the monofunctional monomer(s), multifunctional monomer(s), chain transfer agent(s) and initiator were added to a polymerisation solvent, at a designated overall concentration, in a 500 mL round bottomed flask fitted with a condenser and an overhead stirrer. The solution was then heated, typically to solvent reflux temperature, during this period further aliquot of initiator was added and stirring and heating was continued for a total of eighteen hours, unless otherwise stated. The solutions were then cooled to ambient temperature prior to characterisation

### Characterisation:

Triple Detection-Size Exclusion Chromatography was performed on a Viscotek triple detection instrument. The columns used were two ViscoGel HHR—H columns and a guard column with an exclusion limit for polystyrene of 10⁷ g·mol⁻¹.

THF was the mobile phase, the column oven temperature was set to 35° C., and the flow rate was 1 mL·min⁻¹. The samples were prepared for injection by dissolving 10 mg of polymer in 1.5 mL of HPLC grade THF and filtered of with an Acrodisc® 0.2 μm PTFE membrane. 0.1 mL of this mixture was then injected, and data collected for 30 minutes. Omnisec was used to collect and process the signals transmitted from the detectors to the computer and to calculate the molecular weight.

### ABBREVIATIONS

### Monofunctional Monomers:

BA—n-Butyl acrylate
DMA—dimethylaminoethyl(meth)acrylate
HPMA—2-Hydroxypropyl methacrylate

### St—Styrene

MAA—Methacrylic acid
VP—4-Vinyl pyridine

### Multifunctional Monomers:

EGDMA—Ethyleneglycol dimethacrylate

### Chain Transfer Agents:

DDT—Dodecyl mercaptane

### 2ME—2-Mercaptoethanol

3 MPA—3-Mercaptopropionic acid

### Initiators:

DTBPO—Di-tert-butyl peroxide
TBEC=t-butylperoxy-2-ethylhexyl carbonate (Luperox TBEC)
P=t-butylperoxybenzoate (luperox P)

TABLE 2
Examples 1 to 19.

| | | | | | | | | | solvent/ |
|---|---|---|---|---|---|---|---|---|---|
| | | | | | | | | initiator | reaction |
| Example | Composition | composition | solids | Mn/kD | Mw/kDa | Mw/Mn | α | conc/type | temp/° C. |
| 1 | ST/BA/HPMA/ | 43/35/20/2/ | 70 | 0.84 | 12 | 14 | 0.48 | DTBPO | 1 (145 |
| | MAA/EGDMA/ | 25/18/17 | | | | | | 2% | deg) |
| | DDT/2ME | | | | | | | | |
| 2 | ST/BA/HPMA/ | 43/35/20/2/ | 75 | 0.68 | 9.5 | 14 | 0.34 | DTBPO | 2 (145 |
| | MAA/EGDMA/ | 25/18/17 | | | | | | 2% | deg) |
| | DDT/2ME | | | | | | | | |
| 3 | ST/BA/HPMA/ | 43/35/20/2/ | 80 | 0.6 | 20.3 | 37 | 0.72 | DTBPO | 3 (145 |
| | MAA/EGDMA/ | 25/18/17 | | | | | | 2% | deg) |
| | DDT/2ME | | | | | | | | |
| 4 | ST/BA/DMA/ | 23/47/18/12/ | 68.7 | 34 | 57 | 1.7 | 0.84 | TBEC | 2 (100 |
| | MAA/EGDMA/ | 25/24/11 | | | | | | 8% | deg) |
| | DDT/3MPA | | | | | | | | |
| 5 | ST/BA/DMA/ | 23/47/18/12/ | 75 | 45.5 | 81 | 1.8 | 0.70 | TBEC | 2 (100 |
| | MAA/EGDMA/ | 25/24/11 | | | | | | 8% | deg) |
| | DDT/3MPA | | | | | | | | |
| 6 | ST/BA/DMA/ | 23/47/18/12/ | 80 | 72 | 127 | 1.8 | 0.77 | TBEC | 2 (100 |
| | MAA/EGDMA/ | 25/24/11 | | | | | | 8% | deg) |
| | DDT/3MPA | | | | | | | | |
| 7 | ST/BA/DMA/ | 22/48/18/12/ | 70 | 28 | 46 | 1.6 | | P 8% | 2 (120 |
| | MAA/EGDMA/ | 25/27/13 | | | | | | | deg) |
| | DDT/3MPA | | | | | | | | |
| 8 | ST/BA/DMA/ | 22/48/18/12/ | 75 | 43 | 70 | 1.6 | 0.77 | P 8% | 2 (120 |
| | MAA/EGDMA/ | 25/27/13 | | | | | | | deg) |
| | DDT/3MPA | | | | | | | | |
| 9 | ST/BA/DMA/ | 22/48/18/12/ | 80 | 54 | 117 | 2.2 | | P 8% | 2 (120 |
| | MAA/EGDMA/ | 25/27/13 | | | | | | | deg) |
| | DDT/3MPA | | | | | | | | |
| 10 | VP/ST/ | 25/75/10/15 | 30/70 | 2.3 | 42.9 | 18.7 | 0.46 | TBPO | PGDA |
| | EGDMA/DDT | | | | | | | 1.12 mol | (130) |
| 11 | VP/ST/ | 25/75/10/15 | 27/73 | 6.9 | 61.8 | 9 | 0.42 | TBPO | PGDA |
| | EGDMA/DDT | | | | | | | 1.12 mol | (130) |
| 12 | VP/ST/ | 25/75/10/15 | 30 | 13 | 81.6 | 6.3 | 0.48 | TBPO | PGDA |
| | EGDMA/DDT | | | | | | | 1.12 mol | (130) |
| 13 | VP/ST/ | 25/75/10/15 | 30 | 12 | 77.3 | 6.4 | 0.46 | TBPO | PGDA |
| | EGDMA/DDT | | | | | | | 1.12 mol | (130) |
| 14 | VP/ST/ | 25/75/10/15 | 30 | 23.5 | 63.5 | 2.7 | 0.5 | TBPO | PGDA |
| | EGDMA/DDT | | | | | | | 1.12 mol | (130) |
| 15 | VP/ST/ | 50/50/10/15 | 30 | 8.7 | 45.7 | 5.3 | 0.5 | TBPO | PGDA |
| | EGDMA/DDT | | | | | | | 1.12 mol | (130) |
| 16 | VP/ST/ | 50/50/10/15 | 30 | 16.2 | 45.7 | 2.8 | 0.48 | TBPO | PGDA |
| | EGDMA/DDT | | | | | | | 1.12 mol | (130) |
| 17 | VP/ST/ | 50/50/10/15 | 30 | 10.1 | 32.6 | 3.2 | 0.45 | TBPO | PGDA |
| | EGDMA/DDT | | | | | | | 1.12 mol | (130) |
| 18 | VP/ST/ | 25/75/10/15 | 30 | 2.2 | 21.2 | 9.9 | 0.41 | TBPO | PGDA |
| | EGDMA/DDT | | | | | | | 1.12% at | (148) |
| | | | | | | | | t = 0, | |
| | | | | | | | | 1.12% at | |
| | | | | | | | | t = 4 h | |
| 19 | VP/LMA/ | 85/15/10/15 | 30 | 4.2 | 9 | 2.1 | 0.38 | TBPO | PGDA |
| | EGDMA/DDT | | | | | | | 1.12% at | (148) |
| | | | | | | | | t = 0, | |
| | | | | | | | | 1.12% at | |
| | | | | | | | | t = 4 h | |

Solvents: 1 = 1,2,4-trimethylbenzene, 2 = mixture of xylene (47.9% wt/wt), 1-buthanol (36.2% wt/wt) and 5-methyl-2-hexanone (15.9% wt/wt),

### Measurement of Brookfield Viscosity

Analogues of the traditional linear and “branched” polymers as shown in WO 02/34793 and truly branched versions of these polymers were synthesised according to the details described in WO 02/34793 and the results are indicated in table 3 below.

V1: Acrylic acid₉₉/Stearyl methacrylate₁-Octadecyl mercaptan_{0.2} (Comparative Example—Linear polymer produced in accordance with WO 02/34793)
V2: Acrylic acid₉₉/Stearyl methacrylate₁-Ethyleneglycol dimethacrylate_{1.2}-Octadecyl mercaptan_{0.2} (Comparative Example—“Branched” polymer produced in accordance with WO 02/34793)
V3: Acrylic acid₉₉/Stearyl methacrylate₅-Ethyleneglycol dimethacrylate₅-Octadecyl mercaptan₅ (Branched polymer in accordance with the present invention)
V4: Acrylic acid₉₉/Stearyl methacrylate₁₅-Ethyleneglycol dimethacrylate₁₅-Octadecyl mercaptan₁₅ (Branched polymer in accordance with the present invention)
In these examples, acrylic acid and stearyl methacrylate are the monofunctional monomers, ethyleneglycol dimethacrylate (EGDMA) is the multifunctional monomer or branching agent and octadecylmercaptan is the chain transfer agent.

0.5 w/v % aqueous solutions of each of the polymers were made at pH 7.5 and 20° C. In each case, the Brookfield viscosity (20 rpm, paddle SO₄) was measured as a function of salt concentration, see Table 3.

TABLE 3
Tabulated Brookfield viscosity of the viscosity modifying polymers as a
function of salt concentration.

| | Polymer composition and Brookfield Viscosity | | | |
|---|---|---|---|---|
| [NaCl] | V1 | V2 | V3 | V4 |
| 0.25 | 10 | 40 | 11 | 10 |
| 0.50 | 30 | 60 | 11 | 10 |
| 0.75 | 40 | 80 | 20 | 10 |
| 1.00 | 60 | 90 | 20 | 10 |

Table 3 clearly shows an increase in viscosity for the conventional linear viscosity modifier at increased salt concentrations. The “branched” polymer of WO 02/34793 indicates a more efficient viscosity modification. Truly branched analogues of the polymers of WO 02/34793 according to the present invention showed negligible increases in viscosity on addition of salt. In essence, the “branched” polymers discussed in WO 02/34793 are so lightly branched that they are essentially high molecular weight linear polymers. This is in complete contrast to the copolymers of the present invention.

1. A branched copolymer obtainable by an addition polymerisation process and comprising a hydrophilic component, said polymer comprising:
i) a residue of at least one mono functional monomer comprising one polymerisable double bond per molecule and a molecular weight of less than 1000 Daltons;
ii) a residue of at least one multifunctional monomer comprising at least two polymerisable double bonds per molecule and a molecular weight of less than 1000 Daltons; and wherein the end termini of the copolymer chains comprise one or more of a residue of a chain transfer agent; an initiator or a terminal group derived from a termination reaction;
wherein;
the molar ratio of the mono functional monomer to multifunctional monomer is between 50:1 to 2.5:1 respectively; and
wherein the hydrophilic component is comprised of at least 1 mole % of a combination of a monofunctional monomer, and a multifunctional monomer and/or a chain transfer agent when compared to the total content of monofunctional monomer which is/are comprised of hydrophilic component each with a solubility of 0.18 w/w % in water at 20° C.; and
wherein the residue of the at least one monofunctional monomer with a molecular weight of less than 1000 daltons is selected from the group comprising:
vinyl acids, vinyl acid ester, vinyl aryl compounds, vinyl acid anhydrides, vinyl amides, vinyl ethers, vinyl amines, vinyl aryl amines, vinyl nitriles, vinyl ketones, and derivatives thereof;
hydroxyl-containing monomers and monomers which can be post-reacted to form hydroxyl groups;
acid-containing or acid functional monomers;
zwitterionic monomers;
quaternised amino monomers, oligomeric monomers; and corresponding allyl monomers of the aforesaid vinyl monomers.

2. A branched copolymer according to claim 1 wherein between 1 to 100% mole %, of the at least one monofunctional monomer with a molecular weight of less than 1000 daltons is derived from a hydrophilic monomer.

3. A branched copolymer according to claim 1 wherein at least 10 mole % of the at least one monofunctional monomer with a molecular weight of less than 1000 Daltons is derived from a hydrophilic monomer.

4. A branched copolymer according to claim 1 wherein at least 20 mole %, of the at least one monofunctional monomer with a molecular weight of less than 1000 Daltons is derived from a hydrophilic monomer.

5. A branched copolymer according to claim 1 wherein the molar concentration of multifunctional monomer relative to the amount of monofunctional monomer is greater than or equal to (≧) 2.

6. A branched copolymer according to claim 1 wherein the molar concentration of multifunctional monomer relative to the amount of monofunctional monomer is 2 to 50.

7. A branched copolymer according to claim 1 wherein the molar concentration of multifunctional monomer relative to the amount of monofunctional monomer is 2 to 15.

8. A branched copolymer according to claim 1, in which the multifunctional monomer comprises a residue of a multifunctional monomer selected from the group comprising di- or multivinyl esters, di- or multivinyl amides, di- or multivinyl aryl compounds and di- or multivinyl alk/aryl ethers.

9. A branched copolymer according to claim 8 wherein the multifunctional monomer comprises a residue of a multifunctional monomer selected from the group comprising a multifunctional monomer containing two or more polymerisable groups where the total weight average molecular weight of the molecule is less than 1000 Da.

10. A branched copolymer according to claim 1 wherein when the residue comprises a chain transfer agent, the chain transfer agent comprises between 0 to 50 mole %, of the copolymer.

11. A branched copolymer according to claim 1 wherein when the residue comprises a chain transfer agent, the residue of the chain transfer agent comprises between 0.05 to 30 mole %, of the copolymer.

12. A branched copolymer according to claim 1, wherein the chain transfer agent is selected from the group comprising: monofunctional and multifunctional thiols, alkyl halides.

13. A branched copolymer according to claim 1 wherein the chain transfer agent is selected from the group comprising: thiolactic acid, thioglycolic acid, thioglycerol, thioethanol, cysteine and cysteamine.

14. A branched copolymer according to claim 1 wherein the chain transfer agent comprises a compound which reduces the molecular weight of a copolymer during a free radical polymerisation reaction.

15. A branched copolymer according to claim 1 wherein the chain transfer agent has a molecular weight of 1000 Daltons or less.

16. A branched copolymer according to claim 1, wherein the residue of the initiator comprises between 0 to 15% w/w of the copolymer based on the total weight of the monomers.

17. A branched copolymer according to claim 1, wherein the residue of the initiator comprises between 0.01 to 10% w/w, of the copolymer based on the total weight of the monomers.

18. A branched copolymer according to claim 1, wherein the initiator is selected from the group comprising: persulphates, redox initiators, peroxides, di-peroxides, dialkylperooxides and peroxybenzoates.

19. A branched copolymer according to claim 1, wherein the initiator is selected from the group comprising dialkylperoxides and peroxybenzoates.

20. A branched copolymer according to claim 1, wherein the hydrophilic branched copolymer is prepared at a conversion rate of greater than or equal to 99%.

21. A branched copolymer according to claim 1 wherein the weight average molecular weight (Mw) of the copolymer is between 5 and 1500 kDa.

22. A branched copolymer according to claim 1 wherein the weight average molecular weight (Mw) of the copolymer is greater than or equal to 20 kDa.

23. A branched copolymer according to claim 1 wherein the weight average molecular weight (Mw) of the copolymer is between 10 to 1500 kDa.

24. A branched copolymer according to claim 1 wherein the hydrophilic monomer with a solubility of 0.18 w/w % in water at 20° C. forming the hydrophilic component of the copolymer is selected from the group comprising: (meth)acrylates, styrenics, (meth)acrylamides, N-vinyl alkamides, vinyl ester, vinyl amides and vinyl alkylates.

25. A branched copolymer according to claim 1 wherein the hydrophilic monomer with a solubility of 0.18 w/w % in water at 20° C. comprising the hydrophilic component of the copolymer is selected from the group comprising: (meth)acrylates, (meth)acrylamide and styrenics.

26. A branched copolymer according to claim 1 comprising (meth)acrylate, (meth)acrylamide, or styrenic-based co-polymers containing a hydrophilic moiety such as an acid, basic, ether, amide or ester group which interact with water through charge or H-bonding.

27. A branched copolymer according to claim 1 wherein the hydrophilic monofunctional monomers are selected from the group comprising: amide-containing monomers such as (meth)acrylamide, [3-((meth)acrylamido)propyl]trimethyl ammonium chloride, 3-(dimethylamino)propyl(meth)acrylamide, 3-[N-(3-(meth)acrylamidopropyl)-N,N-dimethyl]aminopropane sulfonate, methyl(meth)acrylamidoglycolate methyl ether and N-isopropyl(meth)acrylamide; (meth)acrylic acid and derivatives thereof such as (meth)acrylic acid, (meth)acryloyl chloride (or any halide), functionalised oligomeric monomers such as monomethoxy oligo(ethyleneglycol)mono(meth)acrylate, monomethoxy oligo(propyleneglycol)mono(meth)acrylate, monohydroxy oligo(ethyleneglycol)mono(meth)acrylate, monohydroxy oligo(propyleneglycol)mono(meth)acrylate, glycerol mono(meth)acrylate and sugar mono(meth)acrylates such as glucose mono(meth)acrylate; vinyl amines such as aminoethyl(meth)acrylate, dimethylaminoethyl(meth)acrylate, diethylaminoethyl(meth)acrylate, diisopropylaminoethyl(meth)acrylate, mono-t-butylaminoethyl(meth)acrylate, morpholinoethyl(meth)acrylate, vinyl aryl amines such as vinyl aniline, vinyl pyridine, N-vinyl carbazole and monomers which can be post-reacted to form amine groups, such as vinyl formamide; vinyl aryl monomers such as styrene sulfonic acid and vinyl benzoic acid; Vinyl hydroxyl monomers such as hydroxyethyl(meth)acrylate, hydroxy propyl(meth)acrylate, glycerol mono(meth)acrylate and monomers which can be post-functionalised into hydroxyl groups such as vinyl acetate, acetoxystyrene and glycidyl(meth)acrylate; acid-containing monomers such as (meth)acrylic acid, styrene sulfonic acid, vinyl phosphonic acid, vinyl benzoic acid, maleic acid, fumaric acid, itaconic acid, 2-(meth)acrylamido 2-ethyl propanesulfonic acid and mono-2-((meth)acryloyloxy)ethyl succinate; and their respective onium salts, zwitterionic monomers such as (meth)acryloyl oxyethylphosphoryl choline and betaine-containing monomers, such as [2-((meth)acryloyloxy)ethyl]dimethyl-(3˜sulfopropyl)ammonium hydroxide; and quaternised amino monomers such as (meth)acryloyloxyethyltrimethyl ammonium chloride.

28. A branched copolymer according to claim 1 wherein the hydrophilic monofunctional monomers are selected from the group comprising: amide-containing monomers such as (meth)acrylamide, [3-((meth)acrylamido)propyl]trimethyl ammonium chloride, methyl(meth)acrylamidoglycolate methyl ether and N-isopropyl(meth)acrylamide; (meth)acrylic acid and derivatives thereof such as (meth)acrylic acid, (meth)acryloyl chloride (or any halide), functionalised oligomeric monomers such as monomethoxy oligo(ethyleneglycol)mono(meth)acrylate, monomethoxy oligo(propyleneglycol)mono(meth)acrylate, monohydroxy oligo(ethyleneglycol)mono(meth)acrylate, monohydroxy oligo(propyleneglycol)mono(meth)acrylate, vinyl amines such as aminoethyl(meth)acrylate, dimethylaminoethyl(meth)acrylate, diethylaminoethyl(meth)acrylate, vinyl aryl amines such as vinyl pyridine, and monomers which can be post-reacted to form amine groups, such as vinyl formamide; vinyl aryl monomers such as styrene sulfonic acid and vinyl benzoic acid; Vinyl hydroxyl monomers such as hydroxyethyl(meth)acrylate, hydroxy propyl(meth)acrylate, and monomers which can be post-functionalised into hydroxyl groups such as vinyl acetate, acetoxystyrene and glycidyl(meth)acrylate; acid-containing monomers such as (meth)acrylic acid, styrene sulfonic acid, vinyl phosphonic acid, vinyl benzoic acid, maleic acid, fumaric acid, itaconic acid, 2-(meth)acrylamido 2-ethyl propanesulfonic acid and mono-2-((meth)acryloyloxy)ethyl succinate; and their respective onium salts, zwitterionic monomers such as and betaine-containing monomers, such as [2-((meth)acryloyloxy)ethyl]dimethyl-(3-sulfopropyl)ammonium hydroxide; and quaternised amino monomers such as (meth)acryloyloxyethyltrimethyl ammonium chloride.

29. A method of preparing a branched copolymer with a hydrophilic component according to claim 1 by an addition process which comprises forming an admixture of:
a) at least one mono functional monomer;
b) at least 2 mole % of a multifunctional monomer relative to the number of moles of mono functional monomer;
c) a chain transfer agent; and/or
d) an initiator;
as previously defined in relation to claim 1 and subsequently reacting said mixture to form a branched copolymer by a solution process wherein the branched copolymer is prepared at a conversion rate of greater than or equal to 99%.

30. A polymer dispersion or solution of a branched copolymer according to claim 1 wherein the copolymer is dissolved or dispersed in an aqueous or non-aqueous solvent or dispersed in an emulsion.

31. A branched copolymer composition comprising:
i) a branched copolymer with a hydrophilic component from a residue of a hydrophilic monofunctional monomer and a multifunctional monomer and/or a chain transfer agent according to claim 1; and
ii) an aqueous solution or non-aqueous solution or emulsion wherein the branched copolymer is dispersed or dissolved in the aqueous solution, non-aqueous solution or emulsion.

32. A composition according to claim 31 wherein the aqueous solution or aqueous emulsion comprises:
water, a salt solution at varying concentrations, an aqueous co-solvent, an aqueous emulsion or an aqueous solution at pH 0 to 14, at temperatures varying between −20° C. to 140° C.

33. Use of a branched copolymer according to claim 1 or a dispersion, emulsion or composition as claimed in claims 30 or 31 in the petrochemical, agrochemical and pharmaceutical industries and for coatings, inks, adhesives and sealants, construction, fuels or lubricants, electronics, water-purification and water-softening, crystal growth inhibition, sizing or wetting agent, freeze-point depressor, or in the home and personal care industries.
